(19) United States
(12) Patent Application Publication
(10) Pub. No.: US 2026/0097196 A1
(43) Pub. Date: Apr. 9, 2026

(54) HEART ASSISTANCE OR BACK-UP DEVICE

(71) Applicant: BYPA MEDICAL SOLUTIONS, Dijon (FR)

(72) Inventor: Bertrand DE BROSSES, Dijon (FR)

(73) Assignee: BYPA MEDICAL SOLUTIONS, Dijon (FR)

(21) Appl. No.: 19/111,817

(22) PCT Filed: Aug. 28, 2023
(86) PCT No.: PCT/EP2023/073557

§ 371 Date: Mar. 14, 2025

(30) Foreign Application Priority Data
Sep. 14, 2022 (FR) FR2209259

(51) Int. Cl. A61M 60/546; A61M 60/113; A61M 60/117; A61M 60/268; A61M 60/38; A61M 60/441

(52) U.S. Cl. CPC A61M 60/546; A61M 60/113; A61M 60/117; A61M 60/268; A61M 60/38; A61M 60/441; A61M 2230/06

(57) ABSTRACT

A device for the temporary circulatory assistance or back-up of the heart of a patient including: a linear actuator configured to move a membrane in a chamber so as to cause a pulsating fluid flow adapted to support the activity of the heart of the patient, which flow is characterised by a succession of phases of aspiration and phases of ejection of the fluid, a control unit configured to pilot the actuator and control the movement of the membrane, which piloting is carried out while taking into account input data originating from an ECG signal of the patient, the control unit is configured to determine a time offset between the instant at which the QRS complex of the ECG signal is detected and the instant at which an ejection phase is initiated.

### TECHNICAL FIELD

An object of the present invention is a device for assisting or backing up the heart.

The term assistance is used when a portion of the blood arriving at the heart is picked up whereas the term back-up is used when all, or almost all (>90%), of the blood arriving at the heart is picked up by the heart back-up device.

It relates to the technical field of devices used by an extracorporeal circulatory assistance/back-up that maintain life-compatible haemodynamics, in the context of a failure of the heart muscle threatening the vital prognosis of the patient, following an acute or chronic heart failure (a coronary insufficiency and/or a cardiovascular disease, or another related pathology).

### PRIOR ART

The function of the heart is to distribute blood in the organism, to transport oxygen and nutrients necessary for the operation of the different organs and to transport the metabolism wastes to its CO² elimination organ, the lungs, urea and creatinine to the kidneys. It is divided into two portions, the right heart which receives the venous blood (depleted in oxygen) via the venae cavae and which sends it via the right ventricle to the pulmonary artery to the respiratory system (or it is recharged with oxygen and discharged in CO²), and the left heart which receives via the pulmonary veins the oxygenated blood to eject it via the aorta to the different organs. The contractions of the heart muscle (pump) allow generating a pulsed blood flow in the organism. They enable a continuous servo-control of the blood flow rate to the physiological needs of the organism and of each organ.

In the presence of a failure of the heart muscle (for example myocardial infarction), it is necessary to temporarily back up the pumping function of said cardiac muscle by a mechanical circulatory assistance system, the objective being to gain time for the heart to recover.

Among currently used circulatory assistance systems, the extracorporeal circulation devices (abbreviated as ECC) are known allowing short-circuiting the failing heart using a servo-controlled pump placed outside the body, which receives blood at the vena cavae and injects it at the (thoracic) aorta, to mechanically generate a continuous blood flow rate compatible with the vital needs of the organism. The main objective pursued during a circulatory assistance is to regulate the pumped flow rate in full match with the physiological needs of the patient, these continuously varying.

As an energy source, a console or a central unit includes a mathematical model designed based on physical laws governing the movement of a fluid in a closed circuit. This circuit is generally composed of a pump, a heat exchanger, a flow meter, a gas and blood electrolyte analyser, a pressure sensor as well as biocompatible equipment such as the tubing, the arterial and venous cannulas, the venous reservoir, the oxygenator, the arterial filter. Usually, a centrifugal or peristaltic pump is used as an arterial head pump and four other peristaltic pumps are used for cardiotomy aspiration, cardiac chamber circulation, cardioplegia administration and a backup pump. If a failure persists, ECMO (“Extracorporeal Membrane Oxygenation”) or ECLS (“Extracorporeal Life Support”) is prescribed.

These systems are simpler than a standard ECC and are transportable, the usage time being several days unlike conventional ECC. Indeed, unlike conventional ECC, ECMO and ECLS are maintained until cardiopulmonary recovery of the patient or as a relay before a transplantation.

Although these systems have demonstrated their effectiveness in their conventional circulatory assistance functions, they are not perfectly satisfactory to the extent that the servo-control of the generated blood flow rate is not optimum with regards to the physiology of the patient. The current survival rate for some addressed pathologies does not exceed 30%. The bloodstream could be imperfectly synchronised, thereby leading to a rise in the left ventricular post-charge which opposes the “contraction” (ejection) of the heart muscle in systole. A fatigued or failing heart will become more fatigued if the aortic pressure is high during systole: the aortic valves open poorly, the post-charge increases and the tele-diastolic pressure of the left ventricle too with an absence of ejection. A leakage of the mitral valve likely to cause an irreversible pulmonary oedema is also noticed. This is one of the main limitations of the systems of the prior art.

Patent document EP3818996A1 describes an assistance device comprising a linear actuator whose piston is movable in translation in a chamber. The translation of the piston ensures the movement of a membrane and the aspiration and ejection of blood. A controller synchronises piloting of the actuator according to the cardiac cycle of the patient. In particular, the blood ejection phase is delayed as of detection of the QRS complex of the ECG signal. Nonetheless, the time offset is particularly complex and does not allow avoiding a rise in the left ventricular post-charge.

The present invention aims to overcome the aforementioned drawbacks. In particular, it is an objective of the invention to provide an assistance device the operation of which avoids, at each cardiac cycle, a rise in the left ventricular post-charge. Another objective of the invention is to optimise the real-time synchronisation of the ejection phases with regards to the physiology of the patient to preserve life-compatible haemodynamics of said patient. Still another objective of the invention is to provide an assistance device whose servo-control is simple to implement and particularly reliable. An objective of the invention is also to allow for better performances and an enhanced accuracy of the pumping cycles.

### DISCLOSURE OF THE INVENTION

The solution provided by the invention is a device for the temporary circulatory assistance or back-up of the heart of a patient, comprising:
a linear actuator configured to move a membrane in a chamber so as to cause a pulsating fluid flow adapted to support the activity of the heart of said patient, which flow is characterised by a succession of phases of aspiration and phases of ejection of the fluid,
a control unit configured to pilot the actuator and control the movement of the membrane, which piloting is carried out while taking into account input data originating from an ECG signal of the patient,
the control unit is configured to determine a time offset ΔT between the instant T_{R} at which the QRS complex of the ECG signal is detected and the instant at which an ejection phase is initiated;

Furthermore, the control unit is configured to determine, at each cardiac cycle identified in the ECG signal, the time offset ΔT by the following formula:

$$Δ T = E + K . ( {F}_{actual} - {F}_{ref} )$$

where:
   E is a time value;
   K is a coefficient such that K>0;
   F_{actual} corresponds to the measured heart rate of the patient;
   F_{ref} corresponds to a reference heart rate.

The time offset ΔT (corresponding to triggering of the artificial systole) as defined by the formula according to the invention allows simply and reliably ensuring that, at each cardiac cycle, the fluid (blood) is not ejected during the natural systole, but once the latter is completed and the aortic valve is closed. Thus, the ventricle is protected from any overload. Furthermore, taking into consideration the heart rate of the patient as a trigger parameter of the artificial systole allows optimising the real-time synchronisation of the ejection phases with regards to the physiology of the patient. The Applicant has noticed that this device was particularly effective and allows producing a very accurate pulsating flow, in perfect match with the physiological needs of the patient. The noticed performances relate to all or part of the following improvements: improvement of the condition of the patient generally reducing the stay-time of the patient in the hospital, improvement of the circulatory situation and of the perfusion of the organs thanks to the characteristics of pulsating fluid flow which induces a better microcirculation of vital organs, a better vascular compliance, a reduction of an inotropic drug support, where appropriate, an increase in the brain oxygen saturation.

Other advantageous features of the apparatus object of the invention are listed hereinbelow. Each of these additional features may be considered alone or in combination with the remarkable features defined hereinabove. Each of these additional features contributes, where appropriate, to solving specific technical problems defined further before in the description and in which the remarkable features defined hereinabove do not necessarily participate. These additional features may be the subject-matter, where appropriate, of one or more divisional patent applications:

The device according to claim 1, wherein the control unit (40) is configured to pilot the actuator (10) while taking into account input data further originating from an aortic pressure signal, the value of E differing depending on whether a dicrotic wave is detected or not in said aortic pressure signal.

According to one embodiment, the control unit is configured so that if a dicrotic wave is detected in the aortic pressure signal, then E=(T_{D}−T_{R}), where T_{R} is the instant when the R-wave of the QRS complex is detected and T_{D} is the instant when the dicrotic wave is detected.

According to one embodiment, the control unit is configured so that if no dicrotic wave is detected in the aortic pressure signal, then E takes on a fixed value.

According to one embodiment, the value of E is fixed.

According to one embodiment, the value of E is set between 0.22 seconds and 0.27 seconds.

According to one embodiment, the reference heart rate F_{ref} is comprised between 50 bpm and 90 bpm.

According to one embodiment, the value of the reference heart rate F_{ref} is pre-parameterised and fixed.

According to one embodiment, the value of the reference heart rate F_{ref} is variable and/or adjustable.

According to one embodiment, the value of the coefficient K is comprised between 0.01 and 0.05 and whose unit is a squared time unit (in particular the second or one of these fractions and/or multiples).

According to one embodiment, the value of the coefficient K is pre-parameterised and fixed.

According to one embodiment, the value of the coefficient K is variable and/or adjustable.

### BRIEF DESCRIPTION OF THE FIGURES

Other advantages and features of the invention will appear better upon reading the description of a preferred embodiment hereinafter, with reference to the appended drawings, made as indicative and non-limiting examples and wherein:

FIG. 1 is a schematic view of the device for the temporary circulatory assistance or back-up of the heart of a patient according to the invention.

FIG. 2 illustrates a QRS complex in an ECG signal.

FIG. 3 illustrates the variation of the aortic pressure P over time.

FIG. 4 illustrates the determination of ΔT in the case where a dicrotic wave is detected in the aortic pressure signal.

FIG. 5 illustrates the determination of ΔT in the case where a dicrotic wave is not detected in the aortic pressure signal.

### DESCRIPTION OF THE EMBODIMENTS

The device of the invention is intended to be used in a degraded hemodynamic situation directly threatening the vital prognosis of a patient 12 (for example, with a tissue perfusion pressure PF lower than 50 mm Hg). It allows assisting or backing up the heart 13 of the patient 12.

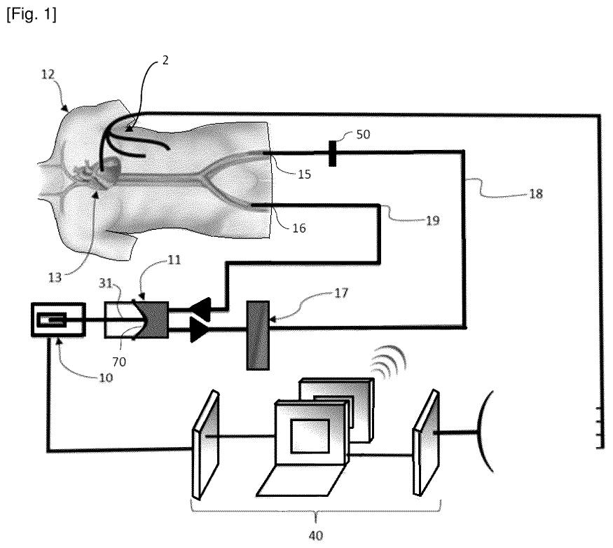

Referring to FIG. 1, the device object of the invention includes a pumping system allowing partial or total back-up of the cardiac muscle by taking in a sufficient amount of blood during the diastole phase of the cardiac cycle and by re-injecting it during the systole phase of said cycle.

The operator introduces an intake cannula 15 (21 or 23 French or “Fr”, FRENCH representing ⅓ of a millimetre) capable of extracting the blood in the venous system of the patient 12 and an ejection cannula 16 (17 or 19 French) capable of injecting the blood into the arterial system of said patient 12. The intake 15 and ejection 16 cannulas commonly used on the extracorporeal circulation market (ECC, ECMO, ECLS) are compatible with the invention. Preferably, armored cannulas are used in order to avoid an admission collapse of the piston, and/or a plication of said cannulas which would result in a reduction in the flow.

The operator can perform:
a left heart/left heart placement for a partial assistance in case of failure of the left heart, by positioning the intake cannula 15 at the right atrium (if the septum is pierced, the left atrium is drained) and the ejection cannula 16 at the abdominal aorta;
a right heart/right heart placement for a partial assistance in case of failure of the right heart, by positioning the intake cannula 15 at a vena cava and the ejection cannula 16 at the pulmonary artery;
a right heart/left heart placement in case of failure of the right heart and of the left heart, by positioning the intake cannula 15 at a vena cava and the ejection cannula 16 at the aorta. In the latter case, the lungs are also short-circuited and an oxygenation system 17 (preferably including a heat exchanger), is placed in the bypass circuit to eliminate CO₂ from the blood and load it with O₂ before re-injecting it into the organism. Advantageously, this oxygenation system 17 is arranged after the reservoir 11, i.e. on the ejection portion 18 of the bypass circuit.

The cannulas 15, 16 may be placed percutaneously, in a cardiac and/or vascular catheterisation lab or in an intensive care unit or by a UMAC SAMU unit (UMAC standing for mobile circulatory assistance unit and SAMU standing to emergency medical assistance service), by introducing them via a peripheral blood vessel and bringing them proximate to the heart 13, at the targeted veins or arteries. They may also be placed surgically in a surgical block, by combined percutaneous puncture implantation and surgical opening of the vessels.

These cannulas 15, 16 are connected to the pumping system by catheter-type tubes, also compatible with those commonly used for an ECC (for example the ⅜th calibre) to form on the one hand the admission portion 19 and the ejection portion 18 of the bypass circuit.

The device includes a chamber 11 forming a reservoir (equivalent to an artificial external ventricle) allowing momentarily storing a volume of fluid (for example: blood, blood substitute, blood+blood substitute). A linear actuator 10 is configured to move a membrane 70 in the chamber 11 so as to cause a pulsating fluid flow which could back up the activity of the heart 13. This flow is characterised by a succession of phases of aspiration and phases of ejection of the fluid.

As example and to give an order of magnitude, for a heart beating at 70 bpm (beats per minute), the duration of the aspiration phase amounts about 0.56 seconds so that a normal aspiration flow rate amounts to about 90 ml/s, i.e. 5 l/min (litre per minute). The duration of the ejection phase amounts to about 0.25 seconds so that a normal ejection rate amounts to about 40 ml/s, i.e. 2 l/min. Thanks to the device according to the invention, it is possible to increase (or possibly reduce) the amounts of aspirated/ejected blood to correspond the closest to the actual operation of a heart and to the needs of the organism.

According to one embodiment, the actuator 10 is of the linear motor type or any other equivalent means (for example a cylinder) allowing moving the membrane 70. According to one embodiment, this membrane 70 is fixed, over its periphery, to the inner wall of the chamber 11. Advantageously, it is made of a flexible and elastic material, an elastomer or other. The membrane 70 accommodates a central insert, not visible in the appended figures, provided with an actuating arm forming a piston 31, this piston being fastened to the actuator 10 thanks to a mechanical engagement, fastening or connection means. According to one embodiment, the actuator 10 is configured to execute the high-speed movement commands in order to adjust the actual movement of the fluid to said commands. For this purpose, it is possible to use a linear motor with a low time constant (advantageously lower than or equal to 10 ms) and capable of generating a significant force (advantageously higher than or equal to 500 N), to which a lever arm system could be added to further accelerate it. The fluid could then be suddenly set in motion and stopped at the selected time.

A control unit 40 is provided to automatically pilot the actuator 10 and control the movement of the membrane 70. This unit 40 may be in the form of a processor, microprocessors, CPU (standing for Central Processing Unit) integrated in a computer, a calculator or a similar means.

Each movement step of the actuator 10 corresponds to an inner volume of the chamber 11. This correspondence between the step of the actuator 10 and the inner volume of the chamber 11 is stored or recorded in a memory area of the unit 40. By controlling the movement of the actuator 10, it is therefore possible to very accurately control the volume of fluid aspirated and ejected by the device during successive aspiration and ejection phases.

According to one embodiment, one or more sensor(s) 2 are placed to acquire an electrocardiogramaignal (ECG signal) which corresponds to an electrical activity of the heart 13. The control unit 40 is configured to pilot the movement of the actuator 10 while taking into account input data originating from this ECG signal. According to a preferred embodiment, the acquisition of the data is carried out in real-time, at a fast frequency of 200 Hz. A fast processing (200 Hz in real-time) of the measurements with embedded behavioural models, allowing sending the signal to the actuation system

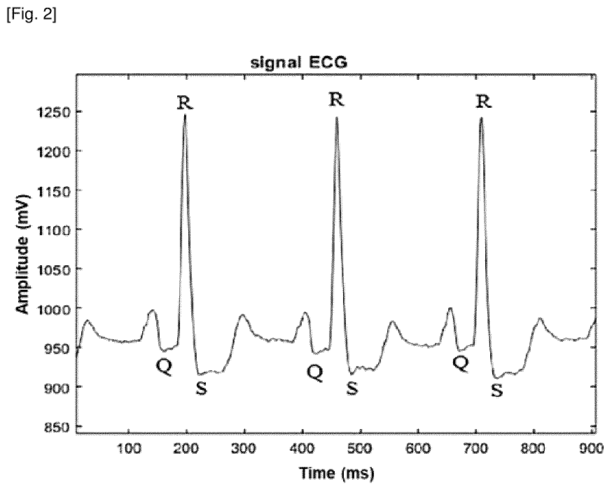

In particular, the control unit 40 is configured to recognise a QRS complex (or QRS wave) as a component of the ECG signal. For example, the unit 40 integrates an algorithm for detecting the QRS complex. Referring to FIG. 2, the QRS complex corresponds to the depolarisation (and to the contraction) of the right and left ventricles. In other words, the QRS complex corresponds to the start of the natural systole. The Q-wave is the first negative wave of the complex. The R-wave is the first positive component of the complex. The S-wave is the second negative component. The shape and the amplitude of the QRS vary according to the branches and according to the possible pathology of the related cardiac muscle. The QRS complex has a normal duration shorter than 0.1 seconds, most often shorter than 0.08 s and its variable amplitude is comprised between 5 mV and 20 mV.

Advantageously, the time at which the QRS complex is detected coincides with the instant of detection of the R-wave to the extent that this wave is the most characteristic of the complex and therefore the simplest to detect. Nonetheless, the instant of detection of the QRS complex could coincide with the instant of detection of the Q-wave or of the S-wave.

According to a feature of the invention, the artificial systole (the phase of ejection of the fluid out of the chamber 11) is not triggered at the same time as the natural systole: a time offset ΔT is induced between the instant T_{R} when the QRS complex of the ECG signal is detected and the instant when the ejection phase is initiated. This time offset ΔT allows ensuring that the aortic valve is perfectly closed when the artificial systole is initiated, so that the ventricle is protected from any overload.

In particular, the unit 40 is configured to determine, at each cardiac cycle identified in the ECG signal, the time offset ΔT by the formula: ΔT=E+K·(F_{actual}−F_{ref}). In this formula, E is a time value whose value is determined further before in the description; K is a coefficient; F_{actual} corresponds to the heart rate of the patient 12; F_{ref} corresponds to a reference heart rate.

The rate F_{actual} is expressed in number of beats per minute (bpm). For simplicity, it is advantageously measured from the ECG signal, but can be measured by means of another apparatus connected to the unit 40, such as for example a blood pressure monitor or a pulse oximeter. Taking the variability of the rate F_{actual} into account in the calculation of the time offset ΔT, allows adapting triggering of the artificial systole to the physiological needs of the patient in real-time.

The best results in terms of performances of the device are obtained when the reference heart rate F_{ref} is comprised between 50 bpm and 90 bpm, preferably equal to 70 bpm. According to one embodiment, the value of F_{ref} is pre-parameterised and fixed. According to another embodiment, the value of F_{ref} is variable and/or adjustable for example according to the age and/or the weight and/or the general condition of the patient 12. According to another embodiment, the value of F_{ref} is determined according to behavioural models embedded in the unit 40.

The difference (F_{actual}−F_{ref}) is equivalent to a correction parameter which depends on the actual activity of the heart 13: the faster the heart, the more the value of ΔT decreases (i.e. the more the artificial systole is rapidly triggered). And vice versa.

K is a coefficient such that K>0 and whose unit is a squared time (in particular the second or one of these fractions and/or multiples). The best results in terms of performances of the device are obtained when the value of K is comprised between 0.01 and 0.05, preferably equal to 0.02. According to one embodiment, the value of K is pre-parameterised and fixed. According to another embodiment, the value of K is variable and/or adjustable for example according to the variation of the rate F_{actual} and/or according to the evolution of the venous pressure and/or the aortic pressure.

According to one embodiment, the value of E is fixed. The best results in terms of performances of the device are obtained when the value of E is comprised between 0.22 seconds and 0.27 seconds, preferably equal to 0.23 seconds. The value of E may also be variable and/or adjustable for example according to the age and/or the weight and/or the general condition of the patient 12 and/or the behavioural models embedded in the unit 40.

According to an advantageous feature of the invention, the unit 40 is configured to pilot the actuator while taking into account as input data not only those originating from the ECG signal, but also those originating from an aortic pressure signal. According to an embodiment illustrated in FIG. 1, this pressure signal originates from a pressure sensor 50 advantageously positioned in the ejection portion 18 of the bypass circuit, which sensor is connected to the unit 40.

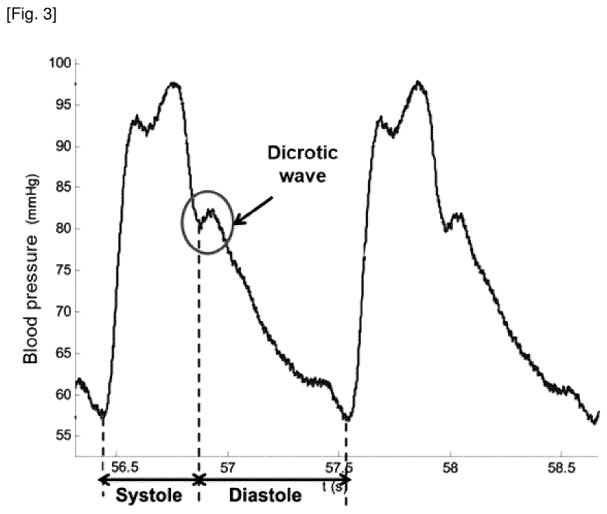

A typical example of variation in the aortic pressure P is illustrated in FIG. 3. The characteristic points and/or phases of such a curve are the following ones:—phase 1: systolic pressure increase; point 2: pressure peak; phase 3: systolic pressure decrease; point 4: dicrotic wave (this “hook”-like rebound corresponds to the closure of the aortic valve); phase 5: diastolic pressure decrease; point 6: telediastolic pressure.

The detection of the dicrotic wave is therefore a major piece of information allowing ensuring that the aortic valve is actually closed. Hence, the unit 40 advantageously integrates an algorithm for detecting a dicrotic wave in the aortic pressure signal. Nonetheless, depending on the condition of the patient and/or the arterial stiffness, this dicrotic wave might be not detected.

Also, according to an advantageous feature of the invention, the value of E differs depending on whether a dicrotic wave is detected or not in the aortic pressure signal.

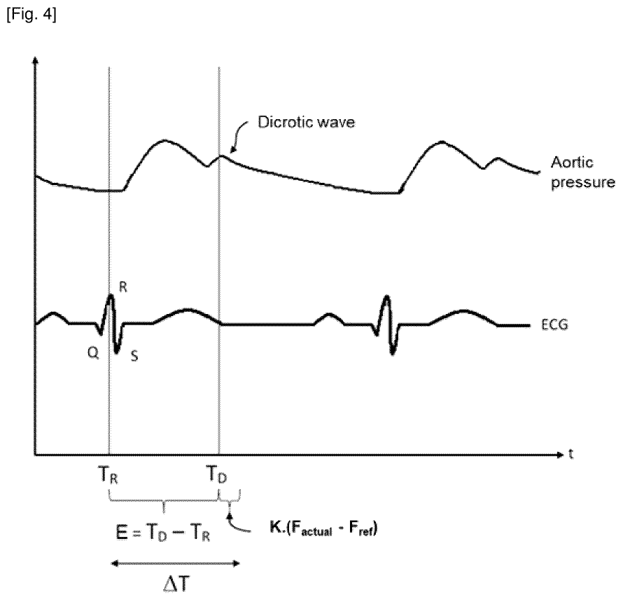

Referring to FIG. 4, if the unit 40 detects a dicrotic wave then E=(T_{D}−T_{R}), where T_{R} is the instant when the R-wave of the QRS complex is detected and T_{D} is the instant when the dicrotic wave is detected. The R-wave being the most characteristic of the complex, it is the easiest one to detect. The invention may also be carried out by detecting the Q-wave or the S-wave of the QRS complex. This first case allows obtaining an optimised operation where triggering of the artificial systole is perfectly synchronised with the closure of the aortic valve and with the heart rate of the patient.

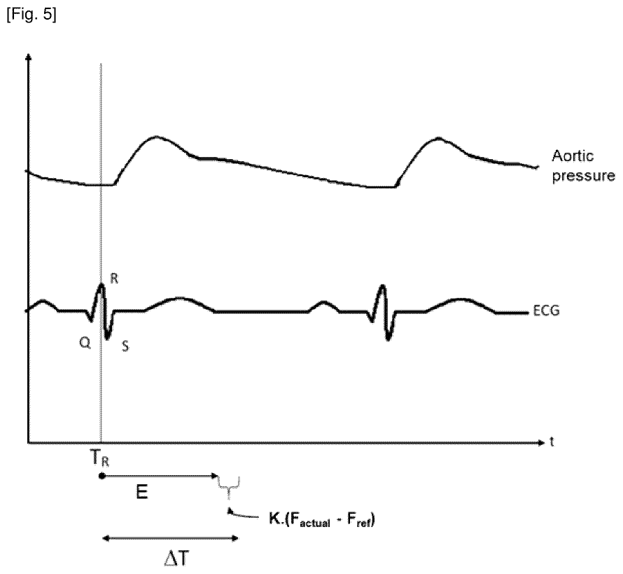

Referring to FIG. 5, if the unit 40 does not detect any dicrotic wave, then E takes on a fixed value, in particular that one mentioned before (between 0.22 seconds and 0.27 seconds, preferably equal to 0.23 seconds). The operation is slightly degraded in comparison with the first case, but still allows triggering the artificial systole while being certain that the aortic valve is closed. If the dicrotic wave is detected at the next cycle or at a subsequent cycle, the determination of E according to the first case applies.

The dual tracking of the QRS complex in the ECG signal and of the dicrotic wave in the aortic pressure signal enables the unit 40 to decide in real-time on triggering of the artificial systole in order to obtain an optimised operation at each cardiac cycle, and particularly robust to the change of the physiological parameters of the patient.

The best results for obtaining an optimum synchronisation between the artificial circulation and the natural heart (beating or not) are obtained by control of the following three elements:

The joint measurement of the ECG signal (QRS complex) and of the dicrotic wave enables an accurate assessment of the condition of the heart. Depending on their availabilities and their qualities, these two measurements allow providing an optimum synchronisation signal, in particular ensuring the preservation of echocardiography and a sinus rhythm.

A fast processing of the measurements (preferably with a fast acquisition frequency of 200 Hz or this frequency magnitude) to send the commands to the actuator 10 in real-time.

An execution of the high-speed commands to adjust the actual movement of the fluid to the commands.

The arrangement of the different elements and/or means and/or steps of the invention, in the above-described embodiments, should not be understood as requiring such an arrangement in all implementations. In any case, it should be understood that various modifications could be made to these elements and/or means and/or steps, without departing from the spirit and the scope of the invention.

Furthermore, one or more feature(s) disclosed solely in one embodiment could be combined with one or more other feature(s) disclosed solely in another embodiment. Similarly, one or more feature(s) disclosed solely in one embodiment could be generalised to the other embodiments, even though this or these feature(s) are described solely in combination with other features.

In any case, in the claims, any reference sign between brackets should not be interpreted as limiting the claim.

1-12. (canceled)

13. A device for the temporary circulatory assistance or back-up of the heart of a patient, comprising:
a linear actuator configured to move a membrane in a chamber so as to cause a pulsating fluid flow adapted to support the activity of the heart of said patient, which flow is characterised by a succession of phases of aspiration and phases of ejection of the fluid,
a control unit configured to pilot the actuator and control the movement of the membrane, which piloting is carried out while taking into account input data originating from an ECG signal of the patient,
the control unit is configured to determine a time offset ΔT between the instant T_{R} at which the QRS complex of the ECG signal is detected and the instant at which an ejection phase is initiated;
wherein the control unit is further configured to determine, at each cardiac cycle identified in the ECG signal, the time offset ΔT by the following formula:
$$Δ T = E + K . ( {F}_{actual} - {F}_{ref} )$$
wherein:
E is a time value;
K is a coefficient such that K>0;
F_{actual} corresponds to the measured heart rate of the patient; and
F_{ref} corresponds to a reference heart rate.

14. The device according to claim 13, wherein the control unit is configured to pilot the actuator while taking into account input data further originating from an aortic pressure signal, the value of E differing depending on whether a dicrotic wave is detected or not in said aortic pressure signal.

15. The device according to claim 14, wherein the control unit is configured so that when a dicrotic wave is detected in the aortic pressure signal, then E=(T_{D}−T_{R}), where T_{R} is the instant when the R-wave of the QRS complex is detected and T_{D} is the instant when the dicrotic wave is detected.

16. The device according to claim 14, wherein the control unit is configured so that when no dicrotic wave is detected in the aortic pressure signal, then E takes on a fixed value.

17. The device according to claim 13, wherein the value of E is fixed.

18. The device according to claim 16, wherein the value of E is set between 0.22 seconds and 0.27 seconds.

19. The device according to claim 13, wherein the reference heart rate F_{ref} is between 50 bpm and 90 bpm.

20. The device according to claim 13, wherein the value of the reference heart rate F_{ref} is pre-parameterised and fixed.

21. The device according to claim 13, wherein the value of the reference heart rate F_{ref} is variable and/or adjustable.

22. The device according to claim 13, wherein the value of the coefficient K is between 0.01 and 0.05 and whose unit is a squared time.

23. The device according to claim 13, wherein the value of the coefficient K is pre-parameterised and fixed.

24. The device according to claim 13, wherein the value of the coefficient K is variable and/or adjustable.

25. The device according to claim 15, wherein the control unit is configured so that when no dicrotic wave is detected in the aortic pressure signal, then E takes on a fixed value.

26. The device according to one of claim 17, wherein the value of E is set between 0.22 seconds and 0.27 seconds.
